(19) United States
(12) Patent Application Publication
(10) Pub. No.: US 2009/0048173 A1
(43) Pub. Date: Feb. 19, 2009

(54) Use Of Dipyridamole For Treatment Of Resistance To Platelet Inhibitors

(76) Inventors: Wolfgang Eisert, Hannover (DE); Victor L. Serebruany, Ellicott City, MD (US)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 11/568,730

(22) PCT Filed: May 10, 2005
(86) PCT No.: PCT/EP05/05024

§ 371 Date: Apr. 11, 2008

(30) Foreign Application Priority Data
May 13, 2004 (US) 60570597

(51) Int. Cl. A61K 38/10; A61K 31/727; G01N 33/567; A61K 31/4709

(52) U.S. Cl. 514/13; 514/56; 514/314; 435/7.21

(57) ABSTRACT

The invention relates to a method of treatment of resistance to platelet inhibitors, i.e. a method to overcome resistance of treatment with platelet inhibitors, said method comprising administering a therapeutically effective amount of dipyridamole in combination with a platelet inhibitor and, optionally, in combination with a third antithrombotic component such as direct thrombin inhibitors, factor Xa inhibitors, combined thrombin/factor Xa inhibitors, heparin, low molecular weight heparin, argatroban, bivalrudin, hirulog or polyglycans to a patient in need thereof. The invention further relates to the use of dipyridamole for the manufacture of a pharmaceutical composition for treatment of resistance to platelet inhibitors. The invention also relates to a method to diagnose resistance to treatment with platelet inhibitors, said method comprising measurement of the density of binding of Annexin V on platelets.

### FIELD OF THE INVENTION

This invention relates to a method of treatment of resistance to platelet inhibitors, i.e. a method to overcome resistance of treatment with currently used platelet inhibitors, said method comprising administering a therapeutically effective amount of dipyridamole (DIP) in combination with a platelet inhibitor and, optionally, in combination with a third antithrombotic component such as direct thrombin inhibitors, factor Xa inhibitors, combined thrombin/factor Xa inhibitors, heparin, low molecular weight heparin, argatroban, bivalrudin, hirulog or polyglycans to a patient in need thereof. The invention further relates to the use of DIP for the manufacture of a pharmaceutical composition for treatment of resistance to platelet inhibitors, i.e. to overcome resistance of treatment with platelet inhibitors. The invention also relates to a method to diagnose resistance to treatment with conventional platelet inhibitors, said method comprising measurement of the density of binding of Annexin V on platelets.

### BACKGROUND OF THE INVENTION

DIP {2,6-bis(diethanolamino)-4,8-dipiperidino-pyrimido[5,4-d]pyrimidine}, closely related substituted pyrimido-pyrimidines and their preparation have been described in e.g. U.S. Pat. No. 3,031,450. DIP was introduced as a coronary vasodilator in the early 1960s. It is also well known having platelet aggregation inhibitor properties due to the inhibition of adenosine uptake. Subsequently, DIP was shown to reduce thrombus formation in a study of arterial circulation of the brain in a rabbit model. These investigations led to its use as an antithrombotic agent; it soon became the therapy of choice for such applications as stroke prevention, maintaining the patency of coronary bypass and valve-replacement, as well as for treatment prior to coronary angioplasty.

Furthermore, the European Stroke Prevention Study 2 (ESPS-2; J Neurol Sci. 1996; 143: 1-13; Neurology 1998; 51: 17-19) proved that treatment by DIP alone was as effective as low-dose aspirin (acetylsalicylic acid; ASA) in the reduction of stroke risk, combination therapy with DIP and ASA was more than twice as effective as ASA alone.

DIP appears to inhibit thrombosis through multiple mechanisms. Early studies showed that it inhibits the uptake of adenosine, which was found to be a potent endogenous anti-thrombotic compound. DIP was also shown to inhibit cyclic AMP phosphodiesterase, thereby increasing intracellular c-AMP.

DIP is a lipophilic compound and has antioxidant properties (Free Radic. Biol. Med. 1995; 18: 239-247) that may contribute to its antithrombotic effect. When oxidized, low density lipoproteins become recognized by the scavenger receptor on macrophages, which is assumed to be the necessary step in the development of atherosclerosis (Ann. Rev. Med. 1992; 43: 219-25).

The inhibition of free radical formation by DIP has been found to inhibit fibrinogenesis in experimental liver fibrosis (Hepatology 1996; 24: 855-864) and to suppress oxygen radicals and proteinuria in experimental animals with aminonucleoside nephropathy (Eur. J. Clin. Invest. 1998; 28: 877-883; Renal Physiol. 1984; 7: 218-226). Inhibition of lipid peroxidation also has been observed in human nonneoplastic lung tissue (Gen. Pharmacol. 1996; 27: 855-859).

In WO 01/30353 is disclosed that fibrin-dependent microcirculation disorders can be treated by DIP, for example microcirculation disorders caused by metabolic diseases, inflammatory reactions or autoimmune diseases, furthermore peripheral microcirculation disorders or microcirculation disorders associated with increased cell fragmentation.

Furthermore, WO 02/085331 discloses that NO-dependent microcirculation disorders can be treated by DIP, due to the activity as free radical scavenger.

WO 02/34248 discloses a method for increasing tissue perfusion with blood by co-administration of an agent that increases cGMP synthesis and an agent that inhibits cGMP degradation in the cells of the blood vessel walls or in blood cells, e.g. by co-administration of a statin and DIP.

The phenomenon of resistance to treatment with platelet inhibitors, e.g. ASA resistance as well as clopidogrel resistance, has been described in literature published especially in the years between 2001 and 2004 (The American Journal of Cardiology, Vol. 88, 230-235, 2001; Journal of the American College of Cardiology, Vol. 41, No. 6, 966-968, 2003; Journal of the American College of Cardiology, Vol. 41, No. 6, 962-965, 2003). It has been described that up to 30% of patients treated with ASA did not show appropriate reduction in platelet aggregability thus being either aspirin resistant or aspirin semiresponders. In the context of the present invention the expression “resistant (or resistance) to platelet-inhibitors” is meant to comprise also semiresponders showing a reduced inhibitory effect on platelet aggregation after administration of e.g. ASA. In most recent literature this phenomenon has also been observed in patients treated with clopidogrel, an ADP-receptor antagonist.

It has been hypothesis and tested that after initial trigger of platelet activation such as through arachidonic acid pathway, which is inhibited by ASA or through the binding of ADP to the appropriate ADP receptor on platelet surfaces, subsequent shape change and changes in the outer membrane produces favorable conditions for the binding of the so called pro thrombinase complex. The prothrombinase complex consisting of clothing factor 5A, 10A and prothrombinase bridged by calcium ion to negatively charged phospholipids lead to an acceleration of the formation of thrombin. This acceleration of thrombin formation has been observed by Hemker et al. (Fibrinolysis, International Journal of Fibrinolysis and Thrombolysis, Abstracts of the Eleventh International Congress of Thrombosis: Ljubljana 1990, Volume 4, Supplement 1, abstract No. 182; Thromb Haemost 62 (1), 1989 abstract No. 1211), who described the increase in Km values for thrombin formation to more than 19,000 times, once the prothrombinase complex has been formed and is bound to negatively charged phospholipids on disturbed membranes. It had been hypothesized, that alterations in the outer cell membrane lead to an increased binding of prothrombinase complexes to the surface and thereby to an increase in thrombin formation which is not modulated by inhibition of either ADP receptor blockade or a modulation of the arachidonic acid pathway within the platelet. In early experiments it could be shown, that the binding of the prothrombinase complex to negatively charged phospholipids could be blocked by Annexin V. Annexin V binding to negatively charged phospholipids inhibits the binding of the prothrombinase complex and thereby inhibits the acceleration of thrombin formation on cell surfaces, thrombin itself being the strongest inducer of platelet aggregation.

It was surprisingly found that patients with resistance to ASA or clopidogrel treatment showed a higher number of binding sites for Annexin V indicating a significantly greater disturbance of the outer membrane of platelets leading to a significant increase of thrombin formation, thus giving raise to elevated production of thrombin which leads to an intrinsic stimulation of platelet activation and subsequent amplification which is not inhibited by conventional inhibitors of platelet activation.

### SUMMARY OF THE INVENTION

Surprisingly, it was found that incubation of cells with DIP showed a significant reduction of Annexin V binding sites compared to pre-incubation in patients with anti-platelet therapy resistance, e.g. ASA or clopidogrel resistance. Reduced formation of Annexin V binding sites reduces excessive formation of thrombin which leads to a insensitivity of platelets to conventional inhibitors of platelet aggregation such as ASA or clopidogrel.

As an explanation it might be assumed that the antioxidative properties of DIP reduce the impact of oxidative as well as metabolic stress to the outer membrane of cells thereby reducing the formation of Annexin V binding sites. Furthermore, it may be that patients with resistance to ASA or clopidogrel treatment show either a genetic or acquired (e.g. dietary acquired) instability of the asymmetry of the outer cell membrane.

Viewed from a first aspect the present invention provides a new approach for a method of treatment of resistance to platelet inhibitors (other than DIP), i.e. a method to overcome resistance of treatment with platelet inhibitors, said method comprising administering a therapeutically effective amount of DIP in combination with a platelet inhibitor and, optionally, in combination with a third antithrombotic component (different from DIP and platelet inhibitors), such as direct thrombin inhibitors, factor Xa inhibitors, combined thrombin/factor Xa inhibitors, heparin, low molecular weight heparin, argatroban, bivalrudin, hirulog or polyglycans, to a patient in need thereof, i.e. a patient resistant to treatment with platelet aggregation inhibitors such as an aspirin resistant, clopidogrel resistant or ticlopidine resistant patient.

Viewed from a second aspect the present invention provides the use of DIP, optionally in combination with a platelet inhibitor and/or a third antithrombotic component (different from DIP and platelet inhibitors), such as direct thrombin inhibitors, factor Xa inhibitors, combined thrombin/factor Xa inhibitors, heparin, low molecular weight heparin, argatroban, bivalrudin, hirulog or polyglycans, for the manufacture of a pharmaceutical composition for treatment of resistance to platelet inhibitors, i.e. to overcome resistance of treatment with platelet inhibitors.

Viewed from a third aspect the present invention provides a method to diagnose resistance to treatment with conventional platelet inhibitors, said method comprising measurement of the density of binding of Annexin V on platelets for identifying platelets with elevated binding of Annexin V to its outer surface as resistant to inhibition of platelet aggregation by conventional platelet inhibitors (such as cyclooxigenase inhibitors, blockers of the receptors known to activate platelets when bound to stimulating ligands (such as ADP receptors) or thrombin receptors, or thromboxane receptors).

The rationale for the combination with a platelet inhibitor clearly is to achieve a successful treatment of the indications the platelet inhibitor normally is given for, e.g. the known prevention therapy of cardiovascular risk patients with the aim to reduce the risk for primary or secondary cardiovascular events. In general, the underlying basic platelet antiaggregatory therapy may be directed to any indication which can be positively influenced by the inhibition of platelets thus, improving the blood supply, especially microcircular blood supply, of affected tissues or organs, including but not limited to

(a) treatment or prevention acute myocardial infarction, prevention of myocardial reinfarction,
(b) treatment or prevention of myocardial ischemia (angina pectoris, ischemic heart diseases, chest pain of ischemic etiology), of coronary heart disease or of acute coronary syndromes, secondary prevention of coronary artery disease, treatment and prevention of recurrent ischemic events after acute myocardial infarction, prevention of left ventricular thrombus formation following anterior myocardial infarction,
(c) treatment or prevention of TIA (transient ischemic attacks, or acute cerebrovascular syndromes), of ischemic stroke or prevention of secondary ischemic stroke,
(d) treatment and prevention of complications of (chronic) atrial fibrillation, e.g. stroke prevention in atrial fibrillation,
(e) reduction of the risk for cardiovascular death,
(f) treatment or prevention of ischemic peripheral circulatory disorders, of peripheral vascular disease or of peripheral microcirculation disorders (e.g. Raynaud's disease, tinnitus or sudden loss of hearing),
(g) treatment or prevention of pulmonary hypertension or of pulmonary embolism, or
(h) treatment or prevention of thromboembolism, acute treatment and extended secondary prevention of deep vein thrombosis (DVT), prevention of venous thromboembolism after major orthopaedic surgery (e.g. hip or knee replacement),
(i) arterial thrombosis of any vessel, peripheral arterial occlusion, retinal vascular accident, catheter thrombotic occlusion or reocclusion, disseminated intravascular coagulation,
(k) prevention of thromboembolic disorders or complications by endovascular procedures, intra-arterial or intravenous lines, implantation of devices, particularly those exposed to the blood flow, such as stents, prosthetic heart valves, filters, etc, whereby this risk of thrombus formation is reduced by the method of the invention, or
(l) prevention of stenosis in vascular grafts, e.g. synthetic vascular grafts, prevention of vascular stent stenosis, prevention of coronary stent stenosis, carotid stent stenosis or peripheral stent stenosis, prevention of stenosis in synthetic grafts used in patients with haemodialysis, prevention of shunt stenosis, prevention of restenosis after angioplasty (e.g. balloon angioplasty, PT(C)A), or preventing reocclusions after bypass operations,
in a person in need thereof, especially a patient resistant to treatment with platelet aggregation inhibitors (other than DIP) having elevated risk in or for said conditions, e.g. elevated risk of cardiovascular events or stroke as may be the case e.g. in diabetic, obese and hypertensive patients or heavy smokers.

The expression “prevention” used hereinbefore should be understood in the sense that the risk to develop a condition mentioned hereinbefore is reduced. The expression “treatment” means therapeutic treatment of patients having already developed one or more of said conditions in manifest form, including symptomatic treatment in order to relieve symptoms of the specific indication or causal treatment in order to reverse or partially reverse the condition or to delay the progression of the indication as far as this may be possible, depending on the condition and the severity thereof.

The method of treatment according to the invention preferably is meant as a combination therapy of a patient resistant to treatment with platelet aggregation inhibitors, comprising a basic therapy with a platelet aggregation inhibitor (other than DIP) and a parallel therapy with DIP in order to achieve the expected antiaggregatory effect or to improve the antiaggregatory effect of the basic therapy. The combination therapy is meant to comprise any parallel treatment regimes with a platelet aggregation inhibitor (other than DIP) and DIP, wherein either DIP or the platelet aggregation inhibitor may be administered first in a sequentiell therapy, or both drugs may be administered simultaneously. In case of parallel treatment including a third antithrombotic component the combination therapy is meant, accordingly, to comprise any parallel treatment regimes, thus sequentiell therapy wherein any of the drugs may be administered as the first, second or third component, furthermore, sequentiell therapy with simultaneous administration of two of the components and earlier or later administration of the third component as well as simultaneous administration of all three components.

The basic therapy is meant to comprise any of the known antiaggregatory therapies with drugs well established for this purpose (with exception of DIP itself, known also to have antiaggregatory activity), such as therapies using platelet inhibitors acting through the arachidonic acid or the ADP pathway. Thus the basic therapy is meant to comprise in a non-limiting manner administration of ASA, clopidogrel, ticlopidine, prasugrel (CS-747, LY640315), cangrelor, AZD-6140, tirofibane, eptifibatide, cilostazol, anagrelide or metabolites thereof having platelet aggregation inhibitory activity. Thus regarding the first and second aspect of the invention any of these platelet aggregation inhibitors may be used, ASA and clopidogrel being preferred.

### DETAILED DESCRIPTION OF THE INVENTION

In the method of treatment according to the invention any of the oral DIP retard, instant or the parenteral formulations on the market may be used, the retard formulations being preferred, for instance those available under the brand name Persantin®, or, already in combination with ASA the formulations available under the brand name Asasantin® or Aggrenox®. Suitable DIP retard formulations are disclosed in EP-A-0032562, instant formulations are disclosed in EP-A-0068191 and combinations of ASA with DIP are disclosed in EP-A-0257344 which are incorporated by reference.

The antithrombotics which may be used as a third component within the first and second aspect of the invention are all known in the art and comprise heparin, low molecular weight heparin, argatroban, bivalrudin, hirulog, antithrombotic polygycans, the direct thrombin inhibitors such as
(1) 1-methyl-2-[N-(4-amidinophenyl)-aminomethyl]-benzimidazol-5-yl-carboxylic acid-N-(2-pyridyl)-N-(2-hydroxycarbonylethyl)-amide, described in WO 98/37075, having the structure

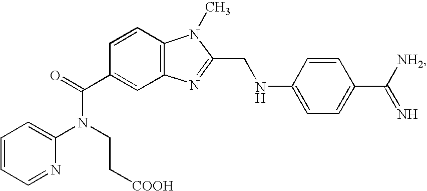

the following prodrug thereof:
(2) dabigatran etexilate(1-methyl-2-[N-[4-(N-n-hexyloxycarbonylamidino)phenyl]-aminomethyl]-benzimidazol-5-yl-carboxylic acid-N-(2-pyridyl)-N-(2-ethoxycarbonyl-ethyl)-amide), also described in WO 98/37075, having the structure

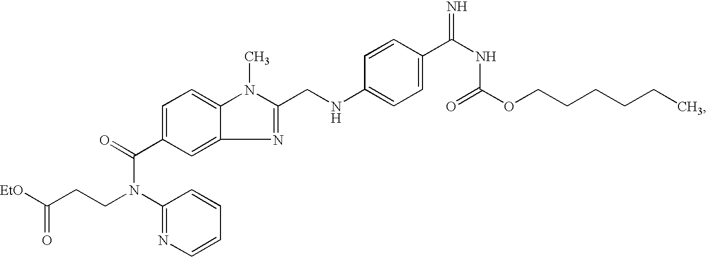

(3) 1-methyl-2-[4-(N-hydroxyamidino)-phenylaminomethyl]-benzimidazol-5-yl-carboxylic acid-(N-2-pyridyl-N-2-ethoxycarbonylethyl)-amide, described in WO 04/014894,
(4) 1-methyl-2-[N-(4-amidinophenyl)-aminomethyl]-benzimidazol-5-yl-carboxylic acid-N-phenyl-N-(2-hydroxycarbonylethyl)-amide (WO 98/37075)
(5) 4-{3-[2,5-dimethyl-4-(N-isopropyl-N-hydroxycarbonylmethylaminocarbonyl-amino)-phenyl]-propargylamino}-benzamidine (DE 199 48 101)
(6) 4-{3-[2,5-dimethyl-4-(N-isopropyl-N-hydroxycarbonylmethylcarbonyl-amino)-phenyl]-propargylamino}-benzamidine (DE 199 48 101)
(7) Melagatran (D. Gustafsson, et al., The Direct Thrombin Inhibitor Melagatran and its Oral Prodrug H 376/95: Intestinal Absorption Properties, Biochemical and Pharmacodynamic Effects, Thromb. Res. 2001, Vol 101 (3), 171-181)

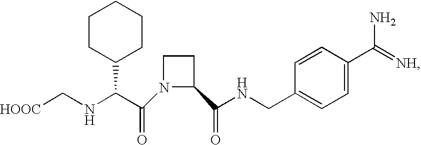

the following orally active prodrug thereof:
(8) Ximelagatran (H-376/95; J. I. Weitz, J. Hirsch; New Anticoagulant Drugs, Chest, 2001, Vol. 119, No. 1 Suppl., 95S-107S)

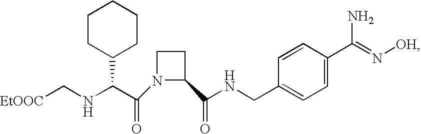

factor Xa inhibitors such as
(9) Razaxaban (DPC-906; Curr Hematol Rep. 2004 September; 3(5): 357-62)
(10) 5-chloro-N—[((5S)-2-oxo-3-[4-(3-oxo-4-morpholinyl)phenyl]-1,3-oxazolidin-5-yl)methyl]-2-thiophencarboxamide (BAY-59-7939, WO 04/60887)
(11) -(indole-6-carbonyl-D-phenylglycinyl)-4-(1-methyl-piperidin-4yl)piperazin (LY-517717, WO02/100847)
(12) 2-(5-carbamimidoyl-2-hydroxy-phenyl)-N-[3-methyl-4-(pyrrolidin-1-yl-carbonyl)-phenyl]-acetamide (WO 03/037220)
(13) 2-(3-carbamimidoyl-phenyl)-N-[3-methyl-4-(pyrrolidin-1-yl-carbonyl)-phenyl]-isobutyramide (WO 02/062748)
(14) 2-(5-carbamimidoyl-2-hydroxy-phenyl)-N-[4-(pyrrolidin-1-yl-carbonyl)-3-trifluoromethyl-phenyl]-propionamide (WO 02/062748)
(15) 2-(3-carbamimidoyl-phenyl)-N-[3-bromo-4-(pyrrolidin-1-yl-carbonyl)-phenyl]-3-(pyridin-4-yl)-propionamide (WO 02/062748)
(16) N-(5-carbamimidoyl-2-hydroxy-benzyl)-3-methyl-4-(pyrrolidin-1-yl-carbonyl)-benzamide (WO 02/062778)
(17) ethyl 2-(3-carbamimidoyl-phenyl)-2-[3-methyl-4-(pyrrolidin-1-yl-carbonyl)-benzoylamino]-acetate (WO 02/062778)
(18) (1) N-(5-Amidino-2-hydroxy-benzyl)-3-trifluormethyl-4-(3-aminomethyl-1,4,5,6-tetrahydro-cyclopentapyrazol-1-yl)-benzamide (WO 02/072558)
(19) 6) N-[1-(5-Amidino-2-hydroxy-phenyl)-ethyl]-3-trifluormethyl-4-(4,5,6,7-tetrahydro-benzimidazol-1-yl)-benzamide (WO 02/072558)
(20) N-(5-Amidino-2-hydroxy-benzyl)-3-trifluormethyl-4-(3-methyl-1,4,5,6-tetrahydro-cyclopentapyrazol-1-yl)-benzamide (WO 02/072558)
(21) 2-(5-amidino-2-hydroxy-phenyl)-N-[3-trifluoromethyl-4-(pyrrolidin-1-yl-carbonyl)-phenyl]-3-phenyl-propionamide (WO 04/013115)
(22) 4-hydroxy-3-{[6-chloro-7-(pyrrolidin-1-yl-carbonyl)-quinazolin-4-yl]aminomethyl}-benzamidine (WO 2004/080970)
(23) 4-hydroxy-3-{[7-methoxy-6-(pyrrolidin-1-yl-carbonyl)-isoquinolin-1-yl]aminomethyl}-benzamidine (WO 2004/080970)
(24) 4-hydroxy-3-{2-phenyl-1-[7-(pyrrolidin-1-yl-carbonyl)-quinazolin-4-ylamino]-ethyl}-benzamidine (WO 2004/080970)
(25) 4-hydroxy-3-{[6-methyl-7-(pyrrolidin-1-yl-carbonyl)-quinazolin-4-yl]aminomethyl}-benzamidine (WO 2004/080970)
(26) 4-hydroxy-3-{[7-(pyrrolidin-1-yl-carbonyl)-quinazolin-4-yl]aminomethyl}-benzamidine (WO 2004/080970)
(27) ethyl 3-(3-amidino-phenyl)-3-{[6-chloro-7-(pyrrolidin-1-yl-carbonyl)-quinazolin-4-yl]amino}-propionate (WO 2004/080970)
(28) 3-(3-amidino-phenyl)-3-{[6-chloro-7-(pyrrolidin-1-yl-carbonyl)-quinazolin-4-yl]amino}-propionic acid (WO 2004/080970)
(29) N-benzoyl-4-hydroxy-3-{[7-(pyrrolidin-1-yl-carbonyl)-quinazolin-4-yl]aminomethyl}-benzamidine (WO 2004/080970)
(30) N-hydroxy-4-hydroxy-3-{[6-methyl-7-(pyrrolidin-1-yl-carbonyl)-quinazolin-4-yl]aminomethyl}-benzamidine (WO 2004/080970)
(31) N-acetoxymethoxycarbonyl-4-hydroxy-3-{[6-methyl-7-(pyrrolidin-1-yl-carbonyl)-quinazolin-4-yl]aminomethyl}-benzamidine (WO 2004/080970)
   and combined thrombin/factor Xa inhibitors, e.g.
(32) 1-methyl-2-[N-(4-amidinophenyl)-aminomethyl]-5-[N-(hydroxycarbonylmethyl)-quinoline-8-sulphonylamino]-benzimidazole (U.S. Pat. No. 6,121,308)
(33) (R)-2-(4-amidinophenylaminomethyl)-1-methyl-5-[1-(carboxymethylamino)-1-(pyrrolidinocarbonyl)-ethyl]-benzimidazole (WO 00/01704)
(34) 2-(4-amidinophenylaminomethyl)-1-methyl-5-[1-(carboxymethylaminomethyl)-1-(pyrrolidinocarbonyl)-ethyl]-benzimidazole (WO 01/47896)
(35) (R)-2-[4-(N-phenylcarbonylamidino)-phenylaminomethyl]-1-methyl-5-[1-(n-propyloxycarbonylmethylamino)-1-(pyrrolidinocarbonyl)-ethyl]-benzimidazole (WO 01/47896)
(36) 3-{[6-(N-acetyl-N-cyclopentylamino)-7-methyl-isoquinolin-1-yl]aminomethyl}-4-hydroxy-benzamidine (WO 2004/080970)
   (the following compounds are disclosed in WO 2004/056784)
(37) N-[1-(5-chloro-1H-benzimidazol-2-yl)-ethyl]-3-methyl-4-(2,5-dihydro-pyrrol-1-yl-carbonyl)-benzamide
(38) N-[1-(5-chloro-1H-benzimidazol-2-yl)-ethyl]-3-ethyl-4-(pyrrolidin-1-yl-carbonyl)-benzamide
(39) N-[1-(5-chloro-1H-benzimidazol-2-yl)-ethyl]-3-chloro-4-(2-aminomethyl-pyrrolidin-1-yl-carbonyl)-benzamide
(40) 3-chloro-N-(5-chloro-1H-benzimidazol-2-yl-methyl)-4-(3-oxo-piperazin-1-yl-carbonyl)-benzamide
(41) N-[1-(5-bromo-1H-benzimidazol-2-yl)-ethyl]-3-methyl-4-(pyrrolidin-1-yl-carbonyl)-benzamide
(42) N-[(5-chloro-1H-benzimidazol-2-yl)-phenyl-methyl]-3-methyl-4-(pyrrolidin-1-yl-carbonyl)-benzamide
(43) N-[1-(5-chloro-1H-benzimidazol-2-yl)-3-methyl-butyl]-3-methyl-4-(pyrrolidin-1-yl-carbonyl)-benzamide
(44) (S)—N-[1-(5-chloro-1H-benzimidazol-2-yl)]ethyl-3-methyl-4-(pyrrolidin-1-yl-carbonyl)-benzamide
(45) N-[(1S)-1-(5-chloro-1H-benzimidazol-2-yl)-ethyl]-3-chloro-4-[(2R/S)-2-aminomethyl-pyrrolidin-1-yl-carbonyl)-benzamide
(46) N-[(1S)-1-(5-chloro-1H-benzimidazol-2-yl)-3-methylsulphanyl-propyl]-3-chloro-4-[(2S)-2-(N-tert.-butoxycarbonyl-aminomethyl)-pyrrolidin-1-yl-carbonyl]-benzamide
(47) N-[(1S)-1-(5-chloro-1H-benzimidazol-2-yl)-butyl]-3-chloro-4-[(2S)-2-aminomethyl-pyrrolidin-1-yl-carbonyl]-benzamide
(48) N-[(1S)-1-(5-chloro-1H-benzimidazol-2-yl)-3-methylsulphanyl-propyl]-3-chloro-4-[(2S)-2-aminomethyl-pyrrolidin-1-yl-carbonyl]-benzamide
(49) N-[(1S)-1-(5-chloro-1H-benzimidazol-2-yl)-3-methylsulphinyl-propyl]-3-chloro-4-[(2S)-2-aminomethyl-pyrrolidin-1-yl-carbonyl]-benzamide
(50) N-[(1S)-1-(5-chloro-1H-benzimidazol-2-yl)-3-methylsulphonyl-propyl]-3-chloro-4-[(2S)-2-aminomethyl-pyrrolidin-1-yl-carbonyl]-benzamide
(51) N-[(1S)-5-(benzyloxycarbonylamino)-1-(5-chloro-1H-benzimidazol-2-yl)-pentyl]-3-methyl-4-(pyrrolidin-1-yl-carbonyl)-benzamide
(52) N-[(1S)-1-(5-chloro-1H-benzimidazol-2-yl)-3-phenyl-propyl]-3-methyl-4-(pyrrolidin-1-yl-carbonyl)-benzamide
(53) N-[(1S)-1-(5-chloro-1H-benzimidazol-2-yl)-3-methylsulphanyl-propyl]-3-methyl-4-(pyrrolidin-1-yl-carbonyl)-benzamide
(54) N-[(1S)-3-benzyloxycarbonyl-1-(5-chloro-1H-benzimidazol-2-yl)-propyl]-3-methyl-4-(pyrrolidin-1-yl-carbonyl)-benzamide
(55) N-[(1S)-1-(5-chloro-1H-benzimidazol-2-yl)-3-(pyrrolidin-1-yl-carbonyl)-propyl]-3-methyl-4-(pyrrolidin-1-yl-carbonyl)-benzamide
(56) N-[(1R)-1-(5-chloro-1H-benzimidazol-2-yl)-2-hydroxy-ethyl]-3-methyl-4-(pyrrolidin-1-yl-carbonyl)-benzamide
(57) N-[1-(5-chloro-1H-benzimidazol-2-yl)-2-methoxy-ethyl]-3-methyl-4-(pyrrolidin-1-yl-carbonyl)-benzamide
(58) N-[(1S)-1-(5-chloro-1H-benzimidazol-2-yl)-3-methoxy-propyl]-3-methyl-4-(pyrrolidin-1-yl-carbonyl)-benzamide
(59) N-[(1R)-2-(C-tert.butoxycarbonyl-methyloxy)-1-(5-chloro-1H-benzimidazol-2-yl)-ethyl]-3-methyl-4-(pyrrolidin-1-yl-carbonyl)-benzamide
(60) N-[(1S)-1-(5-chloro-1H-benzimidazol-2-yl)-3-methylsulphinyl-propyl]-3-methyl-4-(pyrrolidin-1-yl-carbonyl)-benzamide
(61) N-[(5-chloro-1H-benzimidazol-2-yl)-phenyl-methyl]-3-methyl-4-(pyrrolidin-1-yl-carbonyl)-benzamide
(62) N-[1-(5-chloro-1H-benzimidazol-2-yl)-phenyl-methyl]-4-(2,5-dihydro-pyrrol-1-yl-carbonyl)-3-methyl-benzamide
(63) N-[(1S)-1-(5-chloro-1H-benzimidazol-2-yl)-3-methylsulphonylamino-propyl]-3-methyl-4-(pyrrolidin-1-yl-carbonyl)-benzamide
(64) N-{(1S)-1-(5-chloro-1H-benzimidazol-2-yl)-3-[3-(2-chloro-ethyl)-ureido]-propyl}-3-methyl-4-(pyrrolidin-1-yl-carbonyl)-benzamide
(65) N-[(1S)-1-(5-chloro-1H-benzimidazol-2-yl)-butyl]-3-methyl-4-(pyrrolidin-1-yl-carbonyl)-benzamide
(66) 3-bromo-N-[(1S)-1-(5-chloro-1H-benzimidazol-2-yl)-3-methylsulphanyl-propyl]-4-(pyrrolidin-1-yl-carbonyl)-benzamide
(67) 3-chloro-N-[(1S)-1-(5-chloro-1H-benzimidazol-2-yl)-3-(methylsulphanyl)-propyl]-4-(pyrrolidin-1-yl-carbonyl)-benzamide
(68) 3-bromo-N-[(1S)-1-(5-chloro-1H-benzimidazol-2-yl)-3-(methylsulphonyl)-propyl]-4-(pyrrolidin-1-yl-carbonyl)-benzamide
(69) 3-bromo-N-[(1S)-1-(5-chloro-1H-benzimidazol-2-yl)-3-methylsulphinyl-propyl]-4-(pyrrolidin-1-yl-carbonyl)-benzamide
(70) 3-chloro-N-[(1S)-1-(5-chloro-1H-benzimidazol-2-yl)-ethyl]-4-[(2R)-2-(methylsulphonylamino-methyl)-pyrrolidin-1-yl-carbonyl]-benzamide
(71) (1R)-3-bromo-N-[1-(5-chloro-1H-benzimidazol-2-yl)-2-hydroxy-ethyl]-4-(2,5-dihydro-pyrrol-1-yl-carbonyl)-benzamide
(72) (1R)-3-methyl-N-[1-(5-chloro-1H-benzimidazol-2-yl)-2-methoxy-ethyl]-4-(2,5-dihydro-pyrrol-1-yl-carbonyl)-benzamide
(73) (1R)-3-chloro-N-[1-(5-chloro-1H-benzimidazol-2-yl)-2-hydroxy-ethyl]-4-(2,5-dihydro-pyrrol-1-yl-carbonyl)-benzamide
(74) N-{(1S)-1-(5-chloro-1H-benzimidazol-2-yl)-3-[(3R,S)-3-dimethylamino-pyrrolidin-1-yl]-carbonyl-propyl}-3-methyl-4-(pyrrolidin-1-yl-carbonyl)-benzamide
(75) N-{(1S)-1-(5-chloro-1H-benzimidazol-2-yl)-3-[(2R)-2-hydroxymethyl-pyrrolidin-1-yl-carbonyl]-propyl}-3-methyl-4-(pyrrolidin-1-yl-carbonyl)-benzamide
(76) N-{(1S)-1-(5-chloro-1H-benzimidazol-2-yl)-3-[(2S)-2-hydroxymethyl-pyrrolidin-1-yl-carbonyl]-propyl}-3-methyl-4-(pyrrolidin-1-yl-carbonyl)-benzamide
(77) N-[(1S)-1-(5-chloro-1H-benzimidazol-2-yl)-3-(2-methyl-2,6-diaza-spiro[3.4]oct-6-yl-carbonyl)-propyl]-3-methyl-4-(pyrrolidin-1-yl-carbonyl)-benzamide
(78) N-{(1S)-3-[(1R)-2-(aminocarbonyl)-pyrrolidin-1-yl-carbonyl]-1-(5-chloro-1H-benzimidazol-2-yl)-propyl}-3-methyl-4-(pyrrolidin-1-yl-carbonyl)-benzamide
(79) N-{(1S)-1-(5-chloro-1H-benzimidazol-2-yl)-3-[(2R)-2-tert.butoxycarbonyl-aminomethyl-pyrrolidin-1-yl-carbonyl]-propyl}-3-methyl-4-(pyrrolidin-1-yl-carbonyl)-benzamide
(80) N-{(1S)-1-(5-chloro-1H-benzimidazol-2-yl)-3-[(3R,S)-hydroxymethyl-pyrrolidin-1-yl)-carbonyl]-propyl}-3-methyl-4-(pyrrolidin-1-yl-carbonyl)-benzamide
(81) N-[(1S)-1-(5-chloro-1H-benzimidazol-2-yl)-3-(1,1-dioxo-1-thiomorpholine-4-yl-carbonyl]-propyl]-3-methyl-4-(pyrrolidin-1-yl-carbonyl)-benzamide
(82) N-[(1S)-1-(5-chloro-1H-benzimidazol-2-yl)-3-[(4-methyl-3-oxo-piperazin-1-yl-carbonyl)-propyl]-3-methyl-4-(pyrrolidin-1-yl-carbonyl)-benzamide
(83) N-[(1R)-1-(5-chloro-1H-benzimidazol-2-yl)-2-methoxy-ethyl]-3-methyl-4-(pyrrolidin-1-yl-carbonyl)-benzamide
(84) 3-chloro-N-[(1R)-1-(5-chloro-1H-benzimidazol-2-yl)-2-methoxy-ethyl]-4-(2,5-dihydro-pyrrol-1-yl-carbonyl)-benzamide
(85) 3-bromo-N-[(1R)-1-(5-chloro-1H-benzimidazol-2-yl)-2-methoxy-ethyl]-4-(pyrrolidin-1-yl-carbonyl)-benzamide
(86) 3-bromo-N-[(1R)-1-(5-chloro-1H-benzimidazol-2-yl)-2-methoxy-ethyl]-4-(2,5-dihydro-pyrrol-1-yl-carbonyl)-benzamide
(87) 3-methyl-N-[(1R)-1-(5-chloro-1H-benzimidazol-2-yl)-2-hydroxy-ethyl]-4-(2,5-dihydro-pyrrol-1-yl-carbonyl)-benzamide
(88) N-{(1S)-1-(5-chloro-1H-benzimidazol-2-yl)-3-[(2S)-2-aminomethyl-pyrrolidin-1-yl-carbonyl]-propyl}-3-methyl-4-(pyrrolidin-1-yl-carbonyl)-benzamide
(89) N-{(1S)-1-(5-chloro-1H-benzimidazol-2-yl)-3-[(2R)-2-aminomethyl-pyrrolidin-1-yl-carbonyl]-propyl}-3-methyl-4-(pyrrolidin-1-yl-carbonyl)-benzamide
(90) 3-chloro-N-[(1R,S)-1-(5-chloro-1H-benzimidazol-2-yl)-ethyl]-4-[(2R)-2-methoxymethyl-pyrrolidin-1-yl-carbonyl]-benzamide
(91) 3-chloro-N-[1-(5-chloro-1H-benzimidazol-2-yl)-ethyl]-4-(3,4,5,6-tetrahydro-2H-[2,3]-bipyridinyl-1-yl-carbonyl)-benzamide
(92) N-[(1R)-1-(5-chloro-1H-benzimidazol-2-yl)-2-methoxy-ethyl]-4-(pyrrolidin-1-yl-carbonyl)-3-trifluoromethyl-benzamide
(93) N-[(1S)-1,3-bis-(5-chloro-1H-benzimidazol-2-yl)-propyl]-3-methyl-4-(pyrrolidin-1-yl-carbonyl)-benzamide
(94) 3-chloro-N-[(1S)-1-(5-chloro-1H-benzimidazol-2-yl)-ethyl]-4-[(2R/S)-2-dimethylaminomethyl-pyrrolidin-1-yl-carbonyl]-benzamide
(95) N-[(1S)-1-(5-chloro-1H-benzimidazol-2-yl)-3-methanesulphonylamino-propyl]-4-(2,5-di hydro-pyrrol-1-yl-carbonyl)-3-methyl-benzamide
(96) N-[(1S)-1-(5-chloro-1H-benzimidazol-2-yl)-butyl]-4-(2,5-dihydro-pyrrol-1-yl-carbonyl)-3-methyl-benzamide
(97) 3-chloro-N-[(1S)-1-(5-chloro-1H-benzimidazol-2-yl)-butyl]-4-(2,5-dihydro-pyrrol-1-yl-carbonyl)-benzamide
(98) 3-bromo-N-[(1S)-1-(5-chloro-1H-benzimidazol-2-yl)-butyl]-4-(2,5-dihydro-pyrrol-1-yl-carbonyl)-benzamide
(99) 4-(N-acetyl-N-cyclopentyl-amino)-N-[(1S)-1-(5-chloro-1H-benzimidazol-2-yl)-2-methylsulphanyl-ethyl]-3-methyl-benzamide
(100) 3-chloro-N-[(1S)-1-(5-chloro-1H-benzimidazol-2-yl)-ethyl]-4-[(2R)-2-(pyrrolidin-1-yl-methyl)-pyrrolidin-1-yl-carbonyl]-benzamide
(101) 3-bromo-N-[(1R)-1-(5-bromo-1H-benzimidazol-2-yl)-2-methoxy-ethyl]-4-(2,5-dihydro-pyrrol-1-yl-carbonyl)-benzamide
(102) 3-bromo-N-[(1R)-1-(5-chloro-1H-benzimidazol-2-yl)-2-ethoxy-ethyl]-4-(2,5-dihydro-pyrrol-1-yl-carbonyl)-benzamide
(103) N-[(1R)-2-allyloxy-1-(5-chloro-1H-benzimidazol-2-yl)-ethyl]-4-(2,5-dihydro-pyrrol-1-yl-carbonyl)-3-methyl-benzamide
(104) 3-bromo-N-[(1R)-1-(5-chloro-1H-benzimidazol-2-yl)-2-prop-2-ynyloxy-ethyl]-4-(2,5-di hydro-pyrrol-1-yl-carbonyl)-benzamide
(105) N-[(1S)-1-(5-chloro-1H-benzimidazol-2-yl)-3-(1H-tetrazol-5-yl)-propyl]-3-methyl-4-(pyrrolidin-1-yl-carbonyl)-benzamide
(106) N-[(1R)-1-(5-chloro-1H-benzimidazol-2-yl)-2-hydroxy-ethyl]-4-(2,5-dihydro-pyrrol-1-yl-carbonyl)-3-trifluoromethyl-benzamide
(107) 3-chloro-N-[(1R)-1-(5-bromo-1H-benzimidazol-2-yl)-2-hydroxy-ethyl]-4-(2,5-dihydro-pyrrol-1-yl-carbonyl)-benzamide
(108) 3-bromo-N-[(1R)-1-(5-bromo-1H-benzimidazol-2-yl)-2-hydroxy-ethyl]-4-(pyrrolidin-1-yl-carbonyl)-benzamide
(109) 3-methyl-N-[(1R)-1-(5-bromo-1H-benzimidazol-2-yl)-2-hydroxy-ethyl]-4-(pyrrolidin-1-yl-carbonyl)-benzamide
   (the following compounds are disclosed in WO 2004-058743)
(110) 6-chloro-4-[1-(5-chloro-1H-benzimidazol-2-yl)-ethylamino]-7-(2-aminomethyl-pyrrolidin-1-yl-carbonyl)-quinazoline
(111) 6-chloro-4-[1-(S)-(5-chloro-1H-benzimidazol-2-yl)-ethylamino]-7-(2,5-dihydropyrrol-1-yl-carbonyl)-quinazoline
(112) 6-chloro-4-[1-(S)-(5-chloro-1H-benzimidazol-2-yl)-ethylamino]-7-(pyrrolidin-1-yl-carbonyl)-quinazoline
(113) 4-[1-(5-chloro-1H-benzimidazol-2-yl)-3-methylsulphanyl-propylamino]-6-methyl-7-(pyrrolidin-1-yl-carbonyl)-quinoline
(114) 4-[1-(5-chloro-1H-benzimidazol-2-yl)-ethylamino]-6-methyl-7-(pyrrolidin-1-yl-carbonyl)-quinoline
(115) 4-[1-(5-chloro-1H-benzimidazol-2-yl)-ethylamino]-6-methyl-7-(3-oxo-piperazin-1-yl-carbonyl)-quinoline
(116) 4-[(1R/S)-1-(5-chloro-1H-benzimidazol-2-yl)-ethylamino]-6-methyl-7-[(2R)-2-aminomethyl-pyrrolidin-1-yl-carbonyl]-quinoline
(117) 4-[1-(5-chloro-1H-benzimidazol-2-yl)-3-methylsulphanyl-propylamino]-6-methyl-7-(3-oxo-piperazin-1-yl-carbonyl)-quinoline
(118) 4-[1-(5-chloro-1H-benzimidazol-2-yl)-3-methanesulphonyl-propylamino]-6-methyl-7-(pyrrolidin-1-yl-carbonyl)-quinoline
(119) 6-chloro-4-[(1S)-1-(5-chloro-1H-benzimidazol-2-yl)-ethylamino]-7-[(2R)-2-aminomethyl-pyrrolidin-1-yl-carbonyl]-quinazoline
(120) 6-chloro-4-[(1S)-1-(5-chloro-1H-benzimidazol-2-yl)-2-hydroxy-ethylamino]-7-(2,5-dihydropyrrol-1-yl-carbonyl)-quinazoline
(121) 6-chloro-4-[(1S)-1-(5-chloro-1H-benzimidazol-2-yl)-2-hydroxy-ethylamino]-7-[(2R)-2-aminomethyl-pyrrolidin-1-yl-carbonyl]-quinazoline
(122) 6-chloro-4-[1-(5-chloro-1H-benzimidazol-2-yl)-3-hydroxycarbonylpropylamino]-7-(pyrrolidin-1-yl-carbonyl)-quinazoline
(123) 6-chloro-4-[1-(5-chloro-1H-benzimidazol-2-yl)-3-benzyloxycarbonylpropyl-amino]-7-(pyrrolidin-1-yl-carbonyl)-quinazoline
(124) 6-chloro-4-[(1S)-1-(5-chloro-1H-benzimidazol-2-yl)-3-methylsulphanyl-propylamino]-7-[(2R)-2-tert.-butyloxycarbonyl-aminomethyl-pyrrolidin-1-yl-carbonyl]-quinazoline
(125) 6-chloro-4-[(1S)-1-(5-chloro-1H-benzimidazol-2-yl)-3-methylsulphanyl-propylamino]-7-(pyrrolidin-1-yl-carbonyl)-quinazoline
(126) 6-chloro-4-[(1S)-1-(5-chloro-1H-benzimidazol-2-yl)-3-methoxy-propylamino]-7-(2,5-dihydropyrrol-1-yl-carbonyl)-quinazoline
(127) 6-chloro-4-[(1S)-1-(5-chloro-1H-benzimidazol-2-yl)-3-methanesulphonyl-propylamino]-7-(pyrrolidin-1-yl-carbonyl)-quinazoline
(128) 6-chloro-4-[(1S)-1-(5-chloro-1H-benzimidazol-2-yl)-3-methylsulphanyl-propylamino]-7-[(2R)-2-aminomethyl-pyrrolidin-1-yl-carbonyl]-quinazoline
(129) 6-chloro-4-[(1S)-1-(5-chloro-1H-benzimidazol-2-yl)-3-methanesulphinyl-propylamino]-7-(pyrrolidin-1-yl-carbonyl)-quinazoline
(130) 6-chloro-4-[(1S)-1-(5-chloro-1H-benzimidazol-2-yl)-3-benzyloxycarbonyl-propylamino]-7-(pyrrolidin-1-yl-carbonyl)-quinazoline
(131) 6-chloro-4-[(1S)-1-(5-chloro-1H-benzimidazol-2-yl)-2-hydroxy-ethylamino]-7-(piperazin-3-on-1-yl-carbonyl)-quinazoline
(132) 6-chloro-4-[(1S)-1-(5-chloro-1H-benzimidazol-2-yl)-3-hydroxycarbonylpropyl-amino]-7-[(2S)-2-aminomethyl-pyrrolidin-1-yl-carbonyl]-quinazoline
(133) 6-chloro-4-[(1S)-1-(5-chloro-1H-benzimidazol-2-yl)-3-methanesulphonyl-propylamino]-7-[(2R)-2-tert.-butyloxycarbonyl-aminomethyl-pyrrolidin-1-yl-carbonyl]-quinazoline
(134) 6-chloro-4-[(1S)-1-(5-chloro-1H-benzimidazol-2-yl)-3-methanesulphonyl-propylamino]-7-[(2R)-2-aminomethyl-pyrrolidin-1-yl-carbonyl]-quinazoline
(135) 6-chloro-4-[(1S)-1-(5-chloro-1H-benzimidazol-2-yl)-ethylamino]-7-(thiazolidin-3-yl-carbonyl)-quinazoline
(136) 6-chloro-4-[(1S)-1-(5-chloro-1H-benzimidazol-2-yl)-3-ethoxycarbonylpropyl-amino]-7-(pyrrolidin-1-yl-carbonyl)-quinazoline
(137) 4-[(1S)-1-(5-chloro-1H-benzimidazol-2-yl)-ethylamino]-6-methyl-7-(pyrrolidin-1-yl-carbonyl)-quinazoline
(138) 4-[(1S)-1-(5-chloro-1H-benzimidazol-2-yl)-ethylamino]-6-methyl-7-(pyrrolidin-1-yl-carbonyl)-quinazoline
(139) 6-chloro-4-[(1S)-1-(5-chloro-1H-benzimidazol-2-yl)-3-methanesulphinyl-propylamino]-7-[(2R)-2-aminomethyl-pyrrolidin-1-yl-carbonyl]-quinazoline
(140) 4-[(1S)-1-(5-chloro-1H-benzimidazol-2-yl)-3-methylsulphanyl-propylamino]-6-methyl-7-(2,5-dihydropyrrol-1-yl-carbonyl)-quinazoline
(141) 6-bromo-4-[(1S)-1-(5-chloro-1H-benzimidazol-2-yl)-ethylamino]-7-(2,5-dihydropyrrol-1-yl-carbonyl)-quinazoline
(142) 6-chloro-4-[(1S)-1-(5-chloro-1H-benzimidazol-2-yl)-3-ethoxycarbonylpropyl-amino]-7-(2,5-dihydropyrrol-1-yl-carbonyl)-quinazoline
(143) 6-chloro-4-[(1S)-1-(5-chloro-1H-benzimidazol-2-yl)-3-methylsulphanyl-propylamino]-7-(2,5-dihydropyrrol-1-yl-carbonyl)-quinazoline
(144) 6-chloro-4-[(1S)-1-(5-chloro-1H-benzimidazol-2-yl)-butylamino]-7-(2,5-dihydropyrrol-1-yl-carbonyl)-quinazoline
(145) 6-chloro-4-[(1S)-1-(5-chloro-1H-benzimidazol-2-yl)-3-methylsulphanyl-propylamino]-7-(2,5-dihydropyrrol-1-yl-carbonyl)-quinazoline
(146) 6-chloro-4-[(1S)-1-(5-chloro-1H-benzimidazol-2-yl)-2-methoxy-ethylamino]-7-(2,5-dihydropyrrol-1-yl-carbonyl)-quinazoline
(147) 6-chloro-4-[1-(5-chloro-1H-benzimidazol-2-yl)-3-diethylaminocarbonyl-propyl-amino]-7-(pyrrolidin-1-yl-carbonyl)-quinazoline
(148) 6-chloro-4-[1-(5-chloro-1H-benzimidazol-2-yl)-3-[N-methyl-N-piperidin-4-yl-amino]-carbonyl-propyl-amino]-7-(pyrrolidin-1-yl-carbonyl)-quinazoline
(149) 6-chloro-4-[1-(5-chloro-1H-benzimidazol-2-yl)-3-[4-methyl-piperazin-1-yl]-carbonyl-propyl-amino]-7-(pyrrolidin-1-yl-carbonyl)-quinazoline
(150) 6-chloro-4-[1-(5-chloro-1H-benzimidazol-2-yl)-3-(C-piperidin-4-yl-methylamino)-carbonyl-propyl-amino]-7-(pyrrolidin-1-yl-carbonyl)-quinazoline
(151) 6-chloro-4-[1-(5-chloro-1H-benzimidazol-2-yl)-3-(N-benzyl-N-methyl-amino)-carbonyl-propyl-amino]-7-(pyrrolidin-1-yl-carbonyl)-quinazoline
(152) 4-[(1S)-1-(5-chloro-1H-benzimidazol-2-yl)-3-allyloxycarbonylpropyl-amino]-6-methyl-7-(2,5-dihydropyrrol-1-yl-carbonyl)-quinazoline
(153) 6-bromo-4-[(1S)-1-(5-chloro-1H-benzimidazol-2-yl)-3-allyloxycarbonylpropyl-amino]-7-(2,5-dihydropyrrol-1-yl-carbonyl)-quinazoline
(154) 6-chloro-4-[(1S)-1-(5-chloro-1H-benzimidazol-2-yl)-2-methoxy-ethylamino]-7-(pyrrolidin-1-yl-carbonyl)-quinazoline
(155) 6-chloro-4-[(1S)-1-(5-chloro-1H-benzimidazol-2-yl)-3-methanesulphonyl-propylamino]-1-oxy-7-[(2R)-2-aminomethyl-pyrrolidin-1-yl-carbonyl]-quinazoline
(156) 6-chloro-4-[(1S)-1-(5-chloro-1H-benzimidazol-2-yl)-ethylamino]-7-[(2S)-2-(pyrrolidin-1-yl-methyl)-pyrrolidin-1-yl-carbonyl]-quinazoline
(157) 6-chloro-4-[(1S)-1-(5-chloro-1H-benzimidazol-2-yl)-ethylamino]-7-[(2R/S)-2-aminomethyl-thiazolidinyl-carbonyl]-quinazoline
(158) 6-chloro-4-[(1S)-1-(5-chloro-1H-benzimidazol-2-yl)-3-methanesulphonyl-propylamino]-7-[(2R)-2-(methanesulphonyl-aminomethyl)-pyrrolidin-1-yl-carbonyl]-quinazoline
(159) 6-chloro-4-{1-(5-chloro-1H-benzimidazol-2-yl)-3-[(1,2,3,4-tetrahydroiso-quinolin-1-yl)-carbonyl-propyl-amino]}-7-(pyrrolidin-1-yl-carbonyl)-quinazoline
(160) 6-chloro-4-[1-(5-chloro-1H-benzimidazol-2-yl)-3-(benzylamino-carbonyl)-propyl-amino]-7-(pyrrolidin-1-yl-carbonyl)-quinazoline
(161) 6-chloro-4-{1-(5-chloro-1H-benzimidazol-2-yl)-3-[(N-methyl-N-phenethyl-amino-carbonyl)-propyl-amino]}-7-(pyrrolidin-1-yl-carbonyl)-quinazoline
(162) 6-chloro-4-[1-(5-chloro-1H-benzimidazol-2-yl)-3-(hydroxyethylamino-carbonyl)-propyl-amino]-7-(pyrrolidin-1-yl-carbonyl)-quinazoline
(163) 6-chloro-4-{1-(5-chloro-1H-benzimidazol-2-yl)-3-[(C-pyridin-3-yl-methylamino-carbonyl)-propyl-amino]}-7-(pyrrolidin-1-yl-carbonyl)-quinazoline
(164) 6-chloro-4-{1-(5-chloro-1H-benzimidazol-2-yl)-3-[(1-oxa-3,8-diaza-spiro[4.5]decan-2-on-8-yl)-carbonyl]-propyl-amino}-7-(pyrrolidin-1-yl-carbonyl)-quinazoline
(165) 6-chloro-4-[1-(5-chloro-1H-benzimidazol-2-yl)-3-(morpholin-4-yl-carbonyl)-propyl-amino]-7-(pyrrolidin-1-yl-carbonyl)-quinazoline
(166) 6-chloro-4-[1-(5-chloro-1H-benzimidazol-2-yl)-3-(C-cyclohexyl-methylamino-carbonyl)-propyl-amino]-7-(pyrrolidin-1-yl-carbonyl)-quinazoline
(167) 6-chloro-4-[1-(5-chloro-1H-benzimidazol-2-yl)-3-(methoxyethylamino-carbonyl)-propyl-amino]-7-(pyrrolidin-1-yl-carbonyl)-quinazoline
(168) 6-chloro-4-[1-(5-chloro-1H-benzimidazol-2-yl)-3-(dimethylaminoethyl-amino-carbonyl)-propyl-amino]-7-(pyrrolidin-1-yl-carbonyl)-quinazoline
(169) 6-chloro-4-[1-(5-chloro-1H-benzimidazol-2-yl)-3-(cyclopropylamino-carbonyl)-propyl-amino]-7-(pyrrolidin-1-yl-carbonyl)-quinazoline
(170) 6-chloro-4-{(1R/S)-1-(5-chloro-1H-benzimidazol-2-yl)-3-[C-(2R/S)-tetrahydrofuran-2-yl-methylamino-carbonyl)-propyl-amino]}-7-(pyrrolidin-1-yl-carbonyl)-quinazoline
(171) 6-chloro-4-[1-(5-chloro-1H-benzimidazol-2-yl)-3-(dimethylaminopropylamino-carbonyl)-propyl-amino]-7-(pyrrolidin-1-yl-carbonyl)-quinazoline
(172) 6-chloro-4-[1-(5-chloro-1H-benzimidazol-2-yl)-3-(aminoethylamino-carbonyl)-propyl-amino]-7-(pyrrolidin-1-yl-carbonyl)-quinazoline
(173) 6-chloro-4-[1-(5-chloro-1H-benzimidazol-2-yl)-3-(2,2,2-trifluoroethylamino-carbonyl)-propyl-amino]-7-(pyrrolidin-1-yl-carbonyl)-quinazoline
(174) 6-chloro-4-{1-(5-chloro-1H-benzimidazol-2-yl)-3-[N-(2-dimethylamino-ethyl)-N-methyl-amino-carbonyl]-propyl-amino}-7-(pyrrolidin-1-yl-carbonyl)-quinazoline
(175) 6-chloro-4-[1-(5-chloro-1H-benzimidazol-2-yl)-3-(N-piperidin-2-yl-aminocarbonyl)-propyl-amino]-7-(pyrrolidin-1-yl-carbonyl)-quinazoline
(176) 6-chloro-4-{1-(5-chloro-1H-benzimidazol-2-yl)-3-[C-(tetrahydropyran-4-yl)-methylamino-carbonyl]-propyl-amino}-7-(pyrrolidin-1-yl-carbonyl)-quinazoline
(177) 6-chloro-4-[1-(5-chloro-1H-benzimidazol-2-yl)-3-(4-hydroxypiperidin-1-yl-carbonyl)-propyl-amino]-7-(pyrrolidin-1-yl-carbonyl)-quinazoline
(178) 6-chloro-4-{1-(5-chloro-1H-benzimidazol-2-yl)-3-[C-(pyridin-4-yl)-methylamino-carbonyl]-propyl-amino}-7-(pyrrolidin-1-yl-carbonyl)-quinazoline
(179) 6-chloro-4-[1-(5-chloro-1H-benzimidazol-2-yl)-3-(N-methylaminocarbonylmethyl-N-methyl-amino-carbonyl)-propyl-amino]-7-(pyrrolidin-1-yl-carbonyl)-quinazoline
(180) 6-chloro-4-{1-(5-chloro-1H-benzimidazol-2-yl)-3-[N-(2-(1H)-imidazol-4-yl)-ethyl)-N-methyl-amino-carbonyl]-propyl-amino}-7-(pyrrolidin-1-yl-carbonyl)-quinazoline
(181) 6-chloro-4-[1-(5-chloro-1H-benzimidazol-2-yl)-3-(1-thiazolidin-3-yl-carbonyl)-propyl-amino]-7-(pyrrolidin-1-yl-carbonyl)-quinazoline
(182) 6-chloro-4-[1-(5-chloro-1H-benzimidazol-2-yl)-3-(N-cyclopropyl-N-methyl-amino-carbonyl)-propyl-amino]-7-(pyrrolidin-1-yl-carbonyl)-quinazoline
(183) 6-chloro-4-[1-(5-chloro-1H-benzimidazol-2-yl)-3-(N-cyclopropylmethyl-N-methyl-amino-carbonyl)-propyl-amino]-7-(pyrrolidin-1-yl-carbonyl)-quinazoline
(184) 6-chloro-4-[1-(5-chloro-1H-benzimidazol-2-yl)-3-(cyclopentylamino-carbonyl)-propyl-amino]-7-(pyrrolidin-1-yl-carbonyl)-quinazoline
(185) 6-chloro-4-[1-(5-chloro-1H-benzimidazol-2-yl)-3-(N-piperidin-4-yl-aminocarbonyl)-propyl-amino]-7-(pyrrolidin-1-yl-carbonyl)-quinazoline
(186) 6-chloro-4-{1-(5-chloro-1H-benzimidazol-2-yl)-3-[C-(pyridin-2-yl)-methylamino-carbonyl]-propyl-amino}-7-(pyrrolidin-1-yl-carbonyl)-quinazoline
(187) 6-chloro-4-[(1S)-1-(5-chloro-1H-benzimidazol-2-yl)-3-hydroxycarbonyl-propylamino]-7-(pyrrolidin-1-yl-carbonyl)-quinazoline
(188) 6-chloro-4-[(1S)-1-(5-chloro-1H-benzimidazol-2-yl)-ethylamino]-7-(5,6,7,8-tetrahydro-[1,2,4]triazolo[4,3a]pyridin-4-yl)-quinazoline
(189) 6-chloro-4-[(1S)-1-(5-chloro-1H-benzimidazol-2-yl)-3-(1,1-dioxo-isothiazolidin-2-yl)-propyl-amino]-7-(2,5-dihydropyrrol-1-yl-carbonyl)-quinazoline
(190) 6-chloro-4-[(1S)-1-(5-chloro-1H-benzimidazol-2-yl)-3-methanesulphonylamino-propyl-amino]-7-(2,5-dihydropyrrol-1-yl-carbonyl)-quinazoline
(191) 4-[(1S)-1-(5-chloro-1H-benzimidazol-2-yl)-3-(methylsulphanyl)-propylamino]-6-methoxy-7-(2,5-dihydropyrrol-1-yl-carbonyl)-quinazoline
(192) 4-[(1S)-1-(5-chloro-1H-benzimidazol-2-yl)-2-methoxy-ethylamino]-6-methoxy-7-(2,5-dihydropyrrol-1-yl-carbonyl)-quinazoline
(193) 6-chloro-4-[(1S)-1-(5-chloro-1H-benzimidazol-2-yl)-2-methoxy-ethylamino]-7-(thiazolidinyl-carbonyl)-quinazoline
(194) 4-[(1S)-1-(5-chloro-1H-benzimidazol-2-yl)-2-methoxy-ethylamino]-6-methyl-7-(2,5-dihydropyrrol-1-yl-carbonyl)-quinazoline
(195) 4-[(1S)-1-(5-chloro-1H-benzimidazol-2-yl)-2-methoxy-ethylamino]-6-methyl-7-(thiazolidinyl-carbonyl)-quinazoline
(196) 6-bromo-4-[(1S)-1-(5-chloro-1H-benzimidazol-2-yl)-2-methoxy-ethylamino]-7-(2,5-dihydropyrrol-1-yl-carbonyl)-quinazoline
(197) 6-bromo-4-[(1S)-1-(5-chloro-1H-benzimidazol-2-yl)-2-methoxy-ethylamino]-7-(thiazolidinyl-carbonyl)-quinazoline
(198) 6-chloro-4-[(1S)-1-(5-chloro-1H-benzimidazol-2-yl)-ethylamino]-7-(6,7,8,9-tetrahydro-[1,2,4]triazolo[4,3-a]pyridin-4-yl)-quinazoline
(199) 6-chloro-4-{1-(5-chloro-1H-benzimidazol-2-yl)-3-[2-(pyridin-4-yl-amino)-ethylamino-carbonyl]-propylamino}-7-(pyrrolidin-1-yl-carbonyl)-quinazoline
(200) 4-[(1S)-1-(5-bromo-1H-benzimidazol-2-yl)-2-methoxy-ethylamino]-6-chloro-7-(2,5-dihydropyrrolyl-carbonyl)-quinazoline and
(201) 4-[(1S)-1-(5-bromo-1H-benzimidazol-2-yl)-ethylamino]-6-chloro-7-(2,5-dihydropyrrolyl-carbonyl)-quinazoline,
   compounds (1) to (201) being preferred, but compound (2) being especially preferred,
   their stereoisomers such as enantiomers and diastereomers, mixtures of stereoisomers such as racemates, prodrugs, pharmacologically acceptable salts, solvates, e.g. hydrates, and physical modifications thereof, e.g. polymorphs.

Prodrugs of the drugs mentioned above are such derivatives containing one or more groups capable of being cleaved in vivo, particularly a group which can be converted in-vivo into a carboxy group or/and a group capable of being cleaved in vivo from an imino or amino group. Compounds containing two groups capable of being cleaved in vivo are so-called double prodrugs. Groups which can be converted in-vivo into a carboxy group and groups capable of being cleaved in vivo from an imino or amino group are disclosed e.g. in WO 98/37075, being herewith incorporated by reference, as well as in other WO publications cited hereinbefore in connection with specific antithrombotics.

In the method according to the invention a plasma level of DIP of about 0.2 to 5 μmol/L, preferably of about 0.5 to 2 μmol/L or particularly of about 0.8 to 1.5 μmol/L may be maintained. DIP can be administered orally in a daily dosage of 50 to 900 mg, preferably 100 to 700 mg, most preferred 200 to 450 mg, for instance 200 mg twice a day. For long-term treatment it may be of advantage to administer repeated doses such as a dose of 25 mg DIP retard or any other instant release formulation several times a day. For parenteral administration DIP could be given in a dosage of 0.5 to 5 mg/kg body weight, preferably 1 to 3.5 mg/kg body weight, during 24 hours as slow i.v. infusion (not faster than 0.2 mg/min).

### Formulations and Dosages: Platelet Aggregation Inhibitors/ASA

With respect to ASA any of the oral formulations on the market may be used. Reference is made to Rote Liste®2004, Editio Cantor Verlag Aulendorf, Germany, or to Physician's Desk Reference, 58 edition, 2004. This component of the medication may be administered orally in a daily dosage of 10 to 1000 mg, preferably 25 to 400 mg, e.g. 100 to 300 mg, most preferred 30 to 75 mg, for instance 25 mg twice a day.

### Formulations and Dosages: Platelet Aggregation Inhibitors/Clopidogrel

Suitable oral formulations of clopidogrel are disclosed in Rote Liste®2004, Editio Cantor Verlag Aulendorf, Germany, or in Physician's Desk Reference, 58 edition, 2004, and may contain from 25 mg to 500 mg, preferably from 75 mg to 375 mg, and most preferably from 75 mg to 150 mg of clopidogrel. For example, the formulation used may contain 25 mg, 50 mg, 75 mg, 150 mg, 250 mg, or 500 mg of clopidogrel. Clopidogrel may be administered orally in a daily dosage of 10 to 300 mg, preferably 25 to 200 mg, e.g. 50 to 100 mg, for instance 75 mg once a day. Oral administration may be in one or divided doses of two, three, or four times daily. A single daily dose is preferred. Clopidogrel is on the market under the brand names Plavix® and Iscover®.

### Formulations and Dosages: Platelet Aggregation Inhibitors/Ticlopidine

Suitable oral formulations of ticlopidine are disclosed in Rote Liste®2004, Editio Cantor Verlag Aulendorf, Germany, or in Physician's Desk Reference, 58 edition, 2004, and may contain from 25 mg to 600 mg, preferably from 100 mg to 400 mg, and most preferably from 200 mg to 300 mg of ticlopidine. For example, the formulation may contain 25 mg, 50 mg, 75 mg, 150 mg, 250 mg, or 500 mg of ticlopidine. Ticlopidine may be administered orally in a daily dosage of 50 to 1000 mg, preferably 100 to 750 mg, e.g. 250 to 600 mg, for instance 250 mg twice a day. Oral administration may be in one or divided doses of two, three, or four times daily. Preferably administration of two single single doses per day is preferred.

### Formulations and Dosages: Platelet Aggregation Inhibitors/Prasugrel

Suitable oral formulations of prasugrel are disclosed in the literature and may contain from 10 mg to 200 mg, preferably from 20 mg to 100 mg, and most preferably from 30 mg to 80 mg of prasugrel. For example, the formulation may contain 20 mg, 30 mg, 40 mg, 50 mg, 60 mg, 70 mg or 80 mg of prasugrel. Prasugrel may be administered orally in a daily dosage of 10 to 200 mg, preferably 20 to 100 mg, e.g. 30 to 80 mg, for instance 40 or 60 mg once a day. Oral administration may be in one or divided doses of two, three, or four times daily. A single daily dose is preferred.

### Formulations and Dosages: Platelet Aggregation Inhibitors/Cangrelor

Cangrelor is a short-acting injectable platelet inhibitor agent (P2Y12 antagonist) and could be given iv in a dosage of 1-5 μg/kg/min, preferably 2-4 μg/kg/min.

### Formulations and Dosages: Platelet Aggregation Inhibitors/AZD-6140

AZD-6140 is an orally active P2T (ADP) receptor antagonist. Suitable oral formulations of AZD-6140 are disclosed in the literature and may contain from 50 mg to 350 mg, preferably from 100 mg to 300 mg, and most preferably from 150 mg to 250 mg of AZD-6140. For example, the formulation may contain 75 mg, 125 mg, 175 mg, 225 mg, 275 mg or 325 mg of AZD-6140. AZD-6140 may be administered orally in a daily dosage of 50 to 5600 mg, preferably 100 to 300 mg, e.g. 150 to 250 mg, for instance 200 mg once a day. Oral administration may be in one or divided doses of two, three, or four times daily. A single daily dose is preferred.

Formulations and dosages of other platelet aggregation inhibitors are disclosed in the literature, e.g. in Rote Liste®2004, Editio Cantor Verlag Aulendorf, Germany, or in Physician's Desk Reference, 58 edition, 2004.

The antithrombotics mentioned hereinbefore as an optional third component can be administered either in accordance with their usual dosage ranges or, preferably, with a dose below the usual dosage range. The dosage for the antithrombotic in combination with DIP is appropriately 1/50 of the lowest dose normally recommended up to 1/1 of the normally recommended dosage, e.g. 1/20 to 1/2 and preferably 1/10 to 1/2, preferably 1/5 to 1/2. The normally recommended dose for the antithrombotic drug is as follows:

intravenously, preferably administered slowly, or subcutaneously: 0.001 to 3.0 mg/kg body weight (bw) or, preferably, 0.005 to 0.5 mg/kg bw or, more preferred, 0.01 to 0.1 mg/kg bw, once or two times a day, and
orally: 0.03 to 30 mg/kg bw or, preferably, 0.1 to 10 mg/kg bw or, more preferred, 0.1 to 1 mg/kg bw, one to four times a day.

For instance, the normally recommended dose for the antithrombotics (1) to (201) may be the dose disclosed in Rote Liste®2004, Editio Cantor Verlag Aulendorf, Germany, or to Physician's Desk Reference, 58 edition, 2004, e.g. exemplary for melagatran 3 mg/0.3 ml s.c. two times a day, or for ximelagatran 24 mg orally two times a day, or the dose described in the prior art, e.g the references cited in the list of compounds hereinbefore. Suitable formulations of compounds (1) to (201) also are described in the prior art, e.g the references cited in the list of compounds hereinbefore.

The active agents employed in the instant combination therapy can be administered in oral forms as tablets, capsules (each of which includes sustained release or timed release formulations), pills, powders, granules, elixirs, tinctures, suspensions, syrups, and emulsions.

The pharmaceutical compositions to be used according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application. Typical oral formulations include tablets, capsules, syrups, elixirs and suspensions. Typical injectable formulations include solutions and suspensions.

The active drugs can be administered in admixture with pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices. For instance, for oral administration in the form of a tablet or capsule, the active drug component can be combined with a nontoxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing. and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like. The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Any drug mentioned in the context of the invention is meant to comprise also any pharmaceutically acceptable salt, hydrate, polymorph or active metabolite thereof.

The diagnostic method according to the present invention, i.e. determination whether a patient shows resistance to treatment with platelet inhibitors, comprises measurement of the density of binding of Annexin V on platelets obtained from the patient for identifying platelets with elevated binding of Annexin V. The method is characterized by the following steps:
(a) incubation of Annexin V with resting platelets obtained from a patient,
(b) determination of a signal intensity of individual platelets in a standardized fashion providing the amount of bound Annexin V, the signal being provided by a suitable marker for detecting Annexin V binding the Annexin V bound to the platelets is labeled with,
(c) comparing the signal intensity (or amount of Annexin V bound) obtained in step (b) to a control signal intensity (or control amount of bound Annexin V) obtained from platelets of subjects with normal response of platelet aggregometry to standard platelet activation (control platelets).

The Annexin V can be labeled with the marker suitable for detecting Annexin V binding before carrying out incubation step (a) or, in the alternative, after carrying out step (a), wherein the latter case Annexin V already bound to the platelets is labeled with a suitable marker tag. The signal used preferably is a radiation signal, e.g. a fluorescence or radioactive radiation signal, thus the marker may be a fluorescence marker or a radioactive label. In determination step (b) flow cytometry or batch fluorescence may be used.

In one embodiment of the method of diagnosis according to the invention synthetic Annexin V or Annexin V isolated from suitable human or animal tissue (such as human placenta) is labeled with a fluorescent marker and incubated with resting platelets from the patient. Flow cytometry allows quantitative measurement of fluorescence intensity of individual platelet in a standardized fashion. Compared to platelets of healthy subjects with normal response of platelet aggregometry to standard platelet activation (control platelets) platelets from patients with resistance to inhibition by conventional stimuli show almost twice the number of bound labeled Annexin V. Any binding exceeding 1.5 times the control value is found to be either partially or completely resistant to conventional antiplatelet treatment with respect to its platelet activation and aggregation after conventional stimuli such as ADP, collagen, thrombin or thromboxane B2.

### EXAMPLE 1

### Effects of Aggrenox® (25 mg ASA/200 mg DIP) in vitro on Platelet Activation, Annexin-V Binding and Thrombin Generation in Stroke Patients with Aspirin-, or/and Clopidogrel Resistance

Study design: Prospective, non-randomized, single-blinded, pilot, in vitro

Patient Population/Indication:

Serial blood samples from 20 patients after ischemic stroke or TIA who demonstrated aspirin-, or/and clopidogrel resistance. Aspirin/clopidogrel resistance is defined by lack of platelet inhibition after one month of mono- or combination therapy. Lack of platelet inhibition is defined when 4 out of the following 5 parameters are met: ADP-induced platelet aggregation remains >60%; collagen-induced aggregation >70%; whole blood aggregation >18 ohms; expression of GP IIb/IIIa >220 log MFI; and P-selectin cell positivity >8%.

Experiments are done (blood is incubated) with dipyridamole 2 μM/L and 4 μM/L.

Sample size: Blood samples from 20 ASA/Clopidogel resistant patients after ischemic stroke or TIA

TABLE 1
Demographic, Risk Factors and Treatment

| | Non-responders, | |
|---|---|---|
| | n = 20 | |
| Parameter | (aspirin 19, clopidogrel 1) | |
| Age, years | 65.0 ± 8.3 | |
| Sex | | |
| Male | 11 | (55%) |
| Ethnic origin | | |
| Caucasian | 14 | (70%) |
| African-American | 6 | (30%) |
| Diagnosis | | |
| Stroke | 10 | (50%) |
| TIA | 10 | (50%) |
| Risk factors and history | | |
| Smoking history | 11 | (55%) |
| Hypertension | 14 | (70%) |
| Diabetes | 5 | (25%) |
| Previous CAD | 7 | (35%) |
| Peripheral Vascular Disease | 3 | (15%) |
| Medications | | |
| Beta-blockers | 8 | (40%) |
| ACE inhibitors | 8 | (40%) |
| Ca-channel blockers | 9 | (45%) |
| AT-receptor antagonists | 1 | (5%) |
| Diuretics | 4 | (2%) |
| Antidepressants | 6 | (30%) |
| Aspirin | 20 | (100%) |
| Clopidogrel 300 mg | 1 | (5%) |

Stroke characteristics

| | | |
|---|---|---|
| Stroke origin | | |
| Ischemic | 19 | (95%) |
| Hemorrhagic | 0 | (0%) |
| Uncertain | 1 | (5%) |
| Stroke location | | |
| Right hemisphere | 6 | (30%) |
| Left hemisphere | 9 | (45%) |
| Cerebellar | 2 | (10%) |
| Bi-lateral | 0 | |
| Brain Stem | 3 | (15%) |

Twenty ml of blood were collected from each participant, divided into 3 parts, and 2 parts were incubated for 45 min with 2 μM/L and 4 μM/L of Dipyridamole. This concentration corresponds to the physiological Dipyridamole concentration in plasma achieved 0.8-12 hours after oral administration of Aggrenox (25 mg of aspirin+200 mg of Dipyridamole). The third portion was incubated with the vehicle and served as internal control.

Serial data on platelet characteristics are presented at Table 2:

| | | Aspirin/Clopidogrel Non-Responders, n = 20 | | |
|---|---|---|---|---|
| | | (Aspirin 19, Clopidogrel + Aspirin 1) | | |
| | | “Baseline” | | |
| | Aspirin/clopidogrel | (30 days after | Dipyridamole | Dipyridamole |
| Parameter | Responders, n = 20 | treatment) | 2 μg/ml | 4 μg/ml |
| Platelet aggregation | | | | |
| 5 μM ADP (%) | 39.2 ± 14.1 | 65.5 ± 6.3 | 63.6 ± 6.8 | 63.8 ± 6.8 |
| | | | p = 0.13 | p = 0.66 |
| Collagen 1 μM (%) | 20.3 ± 8.4 | 65.9 ± 8.9 | 64.2 ± 7.4 | 64.2 ± 6.9 |
| | | | p = 0.13 | p = 0.38 |
| Arachidonic Acid 0.75 μM | 23.6 ± 6.7 | 72.5 ± 9.5 | 71.4 ± 7.3 | 70.6 ± 6.2 |
| | | | p = 0.41 | p = 0.12 |
| Flow-cytometry | | | | |
| CD41a (GP IIb), MFI | 359.6 + 42.8 | 429.0 ± 52.1 | 417.6 ± 49.5 | 419.4 ± 49.1 |
| | | | p = 0.12 | p = 0.22 |
| CD62p (P-selectin), % + | 5.71 ± 3.4 | 10.0 ± 1.8 | 9.4 ± 2.8 | 9.4 ± 2.4 |
| | | | p = 0.308 | p = 0.21 |
| Thrombin receptor (PAR-1) | 18.9 ± 7.0 | 31.6 ± 6.4 | 27.8 ± 5.4 | 29.1 ± 5.5 |
| SPAN 12 | | | p = 0.02 | p = 0.024 |
| (epitope of intact receptor) | | | | |
| Thrombin receptor (PAR-1) | 13.1 ± 6.6 | 20.2 ± 3.4 | 18.4 ± 3.1 | 18.1 ± 3.0 |
| WEDE 15 | | | p = 0.0507 | p = 0.022 |
| (epitope of cleaved receptors) | | | | |
| Annexin V binding | 4.4 ± 2.8 | 9.5 ± 2.7 | 7.9 ± 2.3 | 7.7 ± 1.9 |
| (PS % positive cells) | | | p = 0.031 | p = 0.022 |
| Thrombin generation markers | | | | |
| D-dimer (μg/L) | 279.1 ± 89.9 | 325.0 ± 84.6 | 316.1 ± 90.1 | 310.8 ± 83.8 |
| | | | p = 0.55 | p = 0.48 |
| Thrombin-antithrombin III | 2.7 ± 1.8 | 3.2 ± 1.1 | 3.2 ± 1.2 | 3.1 ± 1.2 |
| Complex (μg/L) | | | p = 0.49 | p = 0.24 |
| Prothrombin fragment F1 + 2 | 1.7 ± 1.3 | 1.9 ± 1.0 | 1.8 ± 1.0 | 1.8 ± 0.9 |
| (nM/L) | | | p = 0.17 | p = 0.41 |

Methods: Twenty out of 79 patients met all the inclusion criteria: documented evidence of ischemic stroke within the previous 6 months, received at least 81 mg of aspirin for 30 days, and exhibited 4 out of the following 5 laboratory parameters: ADP-induced platelet aggregation >60%; collagen-induced aggregation >70%; whole blood aggregation >18 ohms; expression of GP IIb/IIIa >220 log MFI; and P-selectin cell positivity >8%. Patients on other antithrombotic agents including COX inhibitors, and NSAID were excluded. Blood samples were pretreated with dipyridamole (2 mkg/ml, 4 mkg/ml), simulating the therapeutic range, and then incubated for 45 minutes at 37° C. Platelets were assessed by conventional (1 μM collagen, 0.75 μM arachidonic acid, and 5 μM ADP), and whole blood (1 mg/ml collagen) aggregometry; the expression of GP IIb/IIIa, P-selectin, annexin V binding, intact (SPAN12), and cleaved (WEDE15) PAR-1 thrombin receptors by flow cytometry. Markers of TR (D-Dimer, Thrombin-Antithrombin-III Complexes, and Prothrombin Fragment F1+2) were measured in the autologous plasma samples by ELISA.

Results: Pretreatment of blood with DIP resulted in the diminished expression of intact PAR-1 receptor (p=0.02 and p=0.024), and annexin V binding (p=0.031 and p=0.02) after incubation with 2 mkg/ml and 4 mkg/ml of dipyridamole respectively. The statistically significant (p=0.022) decreased activity of the cleaved PAR-1 was observed only after incubation with 4 mkg/ml. Platelet aggregation, and TG markers were not affected by DIP.

Conclusions: Addition of DIP in vitro in AR patients resulted in the sustained blockade of GP IIb/IIIa, PAR-1 receptors, and annexin-V binding, while aggregometry and TR markers were not changed.

1. A method of treatment of resistance to platelet inhibitors comprising administering a therapeutically effective amount of dipyridamole as a first active component in combination with a platelet inhibitor as a second active component to a patient resistant to treatment with platelet aggregation inhibitors.

2. The method of claim 1 comprising administering as a third active component an antithrombotic.

3. The method of claim 2 wherein the antithrombotic is selected from direct thrombin inhibitors, factor Xa inhibitors, combined thrombin/factor Xa inhibitors, heparin, low molecular weight heparin, argatroban, bivalrudin, hirulog and polyglycans.

4. Use of dipyridamole for the manufacture of a pharmaceutical composition for treatment of resistance to platelet inhibitors.

5. The use of claim 4, wherein dipyridamole, as a first active component, is used in combination with a platelet inhibitor, as a second active component.

6. The use of claim 4, wherein dipyridamole, as a first active component, is used in combination with an antithrombotic (other than a platelet inhibitor), as a second active component.

7. The use of claim 4, wherein dipyridamole, as a first active component, is used in combination with a platelet inhibitor, as a second active component, and an antithrombotic, as a third active component.

8. The use of claim 6 or 7, wherein the antithrombotic is selected from direct thrombin inhibitors, factor Xa inhibitors, combined thrombin/factor Xa inhibitors, heparin, low molecular weight heparin, argatroban, bivalrudin, hirulog and polyglycans.

9. A diagnostic method for determination whether a patient shows resistance to treatment with platelet inhibitors, said method comprising measurement of the density of binding of Annexin V on platelets obtained from the patient for identifying platelets with elevated binding of Annexin V.

10. The method of claim 9 comprising the steps of
(a) incubation of Annexin V with resting platelets obtained from a patient,
(b) determination of a signal intensity of individual platelets in a standardized fashion providing the amount of bound Annexin V, the signal being provided by a suitable marker for detecting Annexin V binding the Annexin V bound to the platelets is labeled with,
(c) comparing the signal intensity (or amount of Annexin V bound) obtained in step (b) to a control signal intensity (or control amount of bound Annexin V) obtained from platelets of subjects with normal response of platelet aggregometry to standard platelet activation (control platelets).
